(19) United States
(12) Patent Application Publication
(10) Pub. No.: US 2012/0107270 A1
(43) Pub. Date: May 3, 2012

(54) Immunocytokines In Combination With Anti-ErbB Antibodies For The Treatment Of Cancer

(76) Inventors: Manuela Kaspar, Brugg (CH); Eveline Trachsel, Zurich (CH)

(21) Appl. No.: 13/381,706

(22) PCT Filed: Jun. 25, 2010
(86) PCT No.: PCT/IB10/01629

§ 371 Date: Jan. 23, 2012

Related U.S. Application Data
(60) Provisional application No. 61/221,925, filed on Jun. 30, 2009.

(51) Int. Cl. A61K 38/20; A61P 35/00

(52) U.S. Cl. 424/85.2

(57) ABSTRACT

This invention relates to the treatment of cancer using anti-ErbB antibodies, such as cetuximab or trastuzumab, in combination with antibody-interleukin 2 (IL2) conjugates which target tenascin-C.

This invention relates to the treatment of cancer using a combination of anti-ErbB antibodies and immunocytokines.

Tenascin-C is a large hexameric glycoprotein of the extracellular matrix which modulates cellular adhesion. It is involved in processes such as cell proliferation and cell migration and is associated with changes in tissue architecture as occurring during morphogenesis and embryogenesis as well as under tumorigenesis or angiogenesis.

A strong over-expression of the large isoform of tenascin-C has been reported for a number of tumors [Borsi 1992 supra], and monoclonal antibodies specific for domains A1 and D, respectively, have been extensively characterised in the clinic [Riva P et al. Int J Cancer 1992; 51:7-13, Riva P et al. Cancer Res 1995; 55:5952s-5956s, Paganelli G et al Eur J Nucl Med 1994; 21:314-321, Reardon D A et al. J Clin Oncol 2002; 20:1389-1397, Bigner D D et al. J Clin Oncol 1998; 16:2202-2212.

Human monoclonal antibody fragments specific to tenascin-C are described in WO2006/050834 and shown to bind preferentially to tumour tissue relative to normal tissue. These antibodies are useful, for example, in delivering toxins, such as cytokines, specifically to tumour cells^{24,25}.

The present inventors have discovered that antibody-cytokine conjugates which target tenascin-C exhibit an unexpected synergy with anti-ErbB antibodies, such as cetuximab and trastuzumab, in the treatment of cancer.

An aspect of the invention provides a method of treating cancer comprising:
administering an anti-ErbB antibody and an antibody-interleukin 2 (IL2) conjugate to an individual in need thereof,
wherein the antibody-IL2 conjugate comprises interleukin 2 (IL2) conjugated to an antibody which specifically binds to tenascin-C.

Other aspects of the invention provide an anti-ErbB antibody for use in a method of treating cancer comprising administering an anti-ErbB antibody in combination with an antibody-IL2 conjugate comprising IL2 conjugated to an antibody which specifically binds to tenascin-C to an individual in need thereof; and the use of an anti-ErbB antibody in the manufacture of a medicament for use in a method of treating cancer comprising administering the anti-ErbB antibody in combination with an antibody-IL2 conjugate to an individual in need thereof, wherein said antibody-IL2 conjugate comprising IL2 conjugated to an antibody which specifically binds to tenascin-C.

Other aspects of the invention provide an antibody-IL2 conjugate comprising IL2 conjugated to an antibody which specifically binds to tenascin-C for use in a method of treating cancer comprising administering the antibody-IL2 conjugate in combination with an anti-ErbB antibody to an individual in need thereof and the use of an antibody-IL2 conjugate comprising IL2 conjugated to an antibody which specifically binds to tenascin-C in the manufacture of a medicament for use in a method of treating cancer comprising administering the antibody-IL2 conjugate in combination with the anti-ErbB antibody to an individual in need thereof.

Other aspects of the invention provide a combination of an anti-ErbB antibody and an antibody-IL2 conjugate comprising IL2 conjugated to an antibody which specifically binds to tenascin-C for use in a method of treating cancer comprising administering the antibody-IL2 conjugate and the anti-ErbB antibody to an individual in need thereof, and the use of a combination of an anti-ErbB antibody and an antibody-IL2 conjugate comprising IL2 conjugated to an antibody which specifically binds to tenascin-C in the manufacture of a medicament for use in a method of treating cancer comprising administering the antibody-IL2 conjugate and the anti-ErbB antibody to an individual in need thereof.

Cancers suitable for treatment as described herein include any type of solid or non-solid cancer or malignant lymphoma and especially leukaemia, sarcomas, skin cancer, bladder cancer, breast cancer, uterine cancer, ovarian cancer, prostate cancer, lung cancer, colorectal cancer, cervical cancer, liver cancer, head and neck cancer, including non-small cell lung cancer, oesophageal cancer, pancreatic cancer, renal cancer, stomach cancer and cerebral cancer. Cancers may be familial or sporadic.

An anti-ErbB antibody binds to a member of the human epidermal growth factor receptor (hEGFR) family, such as epidermal growth factor receptor (EGFR; also known as ErbB-1 or HER-1: Gene ID 1956: Genbank accession number NP_{—}005219), HER-2 (also known as ErbB-2 or neu: GeneID 2064: Genbank accession number NP_{—}001005862), HER-3 (also known as ErbB-3: GeneID 2065: Genbank accession number NP_{—}001973), or HER-4 (also known as ErbB-4: GeneID 2066: Genbank accession number NP_{—}005226).

Various antibodies which bind to EGFR (ErbB-1) are known in the art and are either approved for clinical use or under clinical development, including monoclonal IgG molecules such as cetuximab (Erbitux®), panitumumab (Vectibix®), zalutumumab, nimotuzumab (Theraloc®), and matuzumab.

Cetuximab is a chimeric IgG1 molecule which binds to the extracellular domain of EGFR and inhibits the dimerisation and activation of the receptor [26]. Cetuximab is produced by Merck KGaA.

Panitumumab is a human IgG2 molecule which also binds to the extracellular domain of EGFR. Panitumumab is produced by Amgen Inc, CA USA.

Zalutumumab is a human IgG1 molecule which binds to extracellular domain III of EGFR. Zalutumumab is produced by Genmab A/S, Denmark.

Nimotuzumab is a humanized IgG1 molecule which binds to the extracellular domain of EGFR. Nimotuzumab (Theraloc®) is produced by Oncosciences AG, Germany.

Matuzumab is a humanized IgG1 molecule which binds to the extracellular domain of EGFR. Matuzumab is produced by Takeda Pharmaceutical Co. Ltd and Merck KGaA.

A suitable antibody which binds to EGFR may include cetuximab, panitumumab, zalutumumab, nimotuzumab, or matuzumab or an antibody which competes for binding to EGFR with any of these antibodies.

In some preferred embodiments, when the anti-ErbB antibody is an antibody which binds EGFR, the cancer which is treated may be a cancer which over-expresses EGFR.

In some preferred embodiments, when the anti-ErbB antibody is an antibody which binds EGFR, the cancer which is treated may be colorectal, head and neck cancer, breast, prostate, glioma, ovarian, gastric or lung cancer.

Various antibodies which bind to HER2 (ErbB-2) are known in the art and are either approved for clinical use or under clinical development, including monoclonal IgG molecules such as trastuzumab (Herceptin®) and pertuzumab (Omnitarg®).

Trastuzumab (Herceptin®) is a humanized IgG1 molecule that binds to domain IV of the HER2 receptor [10]. Trastuzumab is produced by Genentech Inc, USA.

Pertuzumab (Omnitarg®) is a humanized IgG1 molecule that binds to domain II of the HER2 receptor [27]. Pertuzumab is produced by Genentech Inc, USA.

A suitable antibody which binds to EGFR may include trastuzumab and pertuzumab or an antibody which competes for binding to EGFR with any of these antibodies.

In some preferred embodiments, when the anti-ErbB antibody is an antibody which binds HER-2, the cancer which is treated may be a cancer which over-expresses EGFR.

In some preferred embodiments, when the anti-ErbB antibody is an antibody which binds HER-2, the cancer which is treated may be breast, ovarian, lung or prostate cancer.

An antibody-IL2 conjugate for use as described herein may comprise interleukin 2 (IL2) conjugated to an antibody which specifically binds to tenascin-C.

Interleukin-2 (IL2) is a secreted cytokine which is involved in immunoregulation and the proliferation of T and B lymphocytes. IL2 has been shown to have a cytotoxic effect on tumour cells and recombinant human IL2 (aldesleukin: Proleukin®) has FDA approval for treatment of metastatic renal carcinoma and metastatic melanoma. The sequence of human IL2 precursor is set out in SEQ ID NO: 11 and publicly available under Genbank database reference NP_{—}000577.2 GI: 28178861.

In some preferred embodiments, the IL2 moiety of the antibody-IL2 conjugate comprises a sequence which has at least 90% sequence identity, at least 95% sequence identity or at least 98% sequence identity to the sequence of mature human IL2, as set out in residues 23-153 of SEQ ID NO: 11.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

In some especially preferred embodiments, the IL2 moiety of the antibody-IL2 conjugate comprises the sequence of mature human IL2 of residues 23-153 of SEQ ID NO: 11.

The IL2 moiety may be fused upstream (N-terminal) or downstream (C-terminal) of the antibody or polypeptide component thereof.

The IL2 moiety may be connected or attached to the antibody moiety of the antibody-IL2 conjugate by any suitable covalent or non-covalent means. In preferred embodiments, the antibody-IL2 conjugate may be a fusion protein comprising IL2 and the anti-tenascin C antibody or a polypeptide component thereof (e.g. a heavy chain or a light chain of an antibody or multi-chain antibody fragment, such as a Fab. Thus, for example, the IL2 moiety may be fused to a VH domain or VL domain of the antibody. Typically the antibody, or component thereof, and IL2 moiety are joined via a peptide linker, e.g. a peptide of about 5-25 residues, e.g. 10-20 residues, preferably about 15 residues. Suitable examples of peptide linkers are well known in the art. In some embodiments, a linker may have an amino acid sequence as set out in SEQ ID NO: 12 or more preferably, a linker may have an amino acid sequence as set out in SEQ ID NO: 17. Normally, the linker has an amino acid sequence comprising one or more tandem repeats of a motif. The motif may be a five residue sequence, and preferably at least 4 of the residues are Gly or Ser. Where four of the five residues is Gly or Ser, the other residue may be Ala. More preferably each of the five residues is Gly or Ser. Preferred motifs are GGGGS, SSSSG, GSGSA and GGSGG. The motif may be a four residue sequence, and preferably at least 3 of the residues are Gly or Ser. Where three of the four residues is Gly or Ser, the other residue may be Ala. More preferably each of the four residues is Gly or Ser. Preferred motifs include GGGS. Preferably, the motifs are adjacent in the sequence, with no intervening nucleotides between the repeats. The linker sequence may comprise or consist of between one and five, preferably three or four, repeats of the motif. For example, a linker with three tandem repeats may have one of the following amino acid sequences:

GGGGSGGGGSGGGGS - SEQ ID NO: 13
SSSSGSSSSGSSSSG - SEQ ID NO: 14
GSGSAGSGSAGSGSA - SEQ ID NO: 15
GGSGGGGSGGGGSGG. - SEQ ID NO: 16

In preferred embodiments, the antibody moiety of the antibody-IL2 conjugate specifically binds to tenascin-C large isoform. For example, the antibody may bind preferentially to tenascin-C large isoform relative to tenascin-C small isoform. Most preferably, the antibody binds to the A1 domain of tenascin-C large isoform.

Preferred antibodies are tumour specific and bind preferentially to tumour tissue relative to normal tissue. Antibodies may, for example, bind to stroma and/or neo- and peri-vascular structures of tumour tissue preferentially to normal tissue.

Examples of suitable antibodies for use in antibody-IL2 conjugates are disclosed in WO2006/050834.

In some embodiments, the antibody moiety of an antibody-IL2 conjugate as described herein competes for binding to tenascin-C with an antibody comprising the 4A1-F16 VH domain of SEQ ID NO. 2 and the 4A1-F16 VL domain of SEQ ID NO. 4.

Competition between antibodies may be assayed easily in vitro, for example using ELISA and/or by tagging a specific reporter molecule to one antibody which can be detected in the presence of other untagged antibody(s), to enable identification of antibodies which bind the same epitope or an overlapping epitope.

A suitable antibody for use in an antibody-IL2 conjugate as described herein may comprise an antibody antigen binding site comprising a VH domain and a VL domain,
the VH domain comprising a VH CDR1 of SEQ ID NO. 5, a VH CDR2 of SEQ ID NO. 6 and a VH CDR3 of SEQ ID NO. 7; and
the VL domain comprising a VL CDR1 of SEQ ID NO. 8, a VL CDR2 of SEQ ID NO. 9 and a VL CDR3 of SEQ ID NO. 10.

In some preferred embodiments, the antibody may comprise an antibody antigen binding site comprising the 4A1-F16 VH domain of SEQ ID NO. 2 and the 4A1-F16 VL domain of SEQ ID NO. 4.

Variants of these VH and VL domains and CDRs may also be employed in antibodies for use in antibody-IL2 conjugates as described herein as described herein. Suitable variants can be obtained by means of methods of sequence alteration or mutation and screening.

Particular variants for use as described herein may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1.

Alterations may be made in one or more framework regions and/or one or more CDRs. In particular, alterations may be made in VH CDR1, VH CDR2 and/or VH CDR3, especially VH CDR3.

Examples of suitable antibody-IL2 conjugates include Teleukin™ (Philogen SpA) and are described in more detail in [24] and [25] below.

Administration of the anti-ErbB antibody, antibody-IL2 conjugate and compositions comprising one or both of these molecules is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors.

The precise dose will depend upon a number of factors, the size and location of the area to be treated, and the precise nature of the anti-ErbB antibody and the antibody-IL2 conjugate (e.g. whole antibody, fragment or diabody). A typical antibody-IL2 conjugate dose will be in the range 0.5 mg to 100 g for systemic applications, and 10 μg to 1 mg for local applications. In some embodiments, the dose of antibody-IL2 conjugate may be up to 22.5 million IU of IL2, administered over a three week cycle.

Typically, the antibody moiety of the conjugate will be a whole antibody, preferably the IgG1 or IgG4 isotype. This is a dose for a single treatment of an adult patient, which may be proportionally adjusted for children and infants, and also adjusted for other antibody formats in proportion to molecular weight. Appropriate doses and regimens for anti-ErbB antibodies are well-known in the art and may be readily determined by a medical practitioner.

Treatments may be repeated at daily, twice-weekly, weekly or monthly intervals, at the discretion of the physician. In some embodiments, treatment may be administered in tri-weekly cycles, with one week of treatment followed by two weeks of recovery.

The antibody-IL2 conjugate and the anti-ErbB antibody may be administered sequentially or simultaneously in accordance with any suitable regimen.

The antibody-IL2 conjugate and the anti-ErbB antibody will usually be administered to an individual in the form of pharmaceutical compositions, which may comprise at least one component in addition to the active compound.

Suitable components include a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may by injection, e.g. intravenous or sub-cutaneous infusion.

For example, for intravenous or sub-cutaneous infusion, the anti-ErbB antibody and the antibody-IL2 conjugate may be in the form of parenterally acceptable aqueous solution(s) which are pyrogen-free and have suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The antibody-IL2 conjugate and the anti-ErbB antibody may be formulated in separate pharmaceutical compositions or, where appropriate, in the same pharmaceutical composition.

Another aspect of the invention provides a pharmaceutical composition for use in the treatment of cancer comprising an anti-ErbB antibody and an antibody-IL2 conjugate comprising interleukin 2 (IL2) conjugated to an antibody which specifically binds to tenascin-C.

Another aspect of the invention provides a method of making a pharmaceutical composition for use in the treatment of cancer comprising formulating an anti-ErbB antibody and an antibody-IL2 conjugate comprising interleukin 2 (IL2) conjugated to an antibody which specifically binds to tenascin-C

Another aspect of the invention provides a therapeutic kit for use in the treatment of cancer comprising an anti-ErbB antibody and an antibody-IL2 conjugate comprising interleukin 2 (IL2) conjugated to an antibody which specifically binds to tenascin-C.

The components of a kit (i.e. the anti-ErbB antibody and antibody-IL2 conjugate) are sterile and in sealed vials or other containers. A kit may further comprise instructions for use of the components in a method described herein. The components of the kit may be comprised or packaged in a container, for example a bag, box, jar, tin or blister pack.

### Terminology

### Antibody

This describes an immunoglobulin whether natural or partly or wholly synthetically produced. The term also covers any polypeptide or protein having a binding domain which is, or is substantially homologous to, an antibody binding domain. Examples of antibodies are the immunoglobulin isotypes and their isotypic subclasses; fragments which comprise an antigen binding domain such as Fab, scFv, Fv, dAb, and Fd; and small immunoproteins (SIPs), minaturised antibodies, camelid VHH domains and diabodies.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH or VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al, Proc. Natl. Acad. Sci. USA 90 6444-6448, 1993). Fv, scFv or diabody molecules may be stabilised by the incorporation of disulphide bridges linking the VH and VL domains (Y. Reiter et al. Nature Biotech 14 1239-1245 1996). Minibodies comprising an scFv joined to a CH3 domain may also be made (S. Hu et al, Cancer Res. 56 3055-3061 1996).

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804).

### Antigen Binding Domain

This describes the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains (e.g. a so-called Fd antibody fragment consisting of a VH domain). Preferably, an antigen binding domain comprises an antibody light chain variable region (VL) and an antibody heavy chain variable region (VH).

### Specific

This may be used to refer to the situation in which one member of a specific binding pair will not show any significant binding to molecules other than its specific binding partner(s). For example, an antibody specific for Tenascin-C may show little or no binding to other components of the extracellular matrix such as fibronectin. Similarly, an antibody specific for Tenascin-C large isoform may show little or no binding to Tenascin-C small isoform. The term is also applicable where e.g. an antigen binding domain is specific for a particular epitope which is carried by a number of antigens, in which case the specific binding member carrying the antigen binding domain will be able to bind to the various antigens carrying the epitope.

### Comprise

This is generally used in the sense of include, that is to say permitting the presence of one or more features or components.

By “substantially as set out” it is meant that the relevant CDR or VH or VL domain of the invention will be either identical or highly similar to the specified regions of which the sequence is set out herein. By “highly similar” it is contemplated that from 1 to 5, preferably from 1 to 4 such as 1 to 3 or 1 or 2, or 3 or 4, substitutions may be made in the CDR and/or VH or VL domain.

The structure for carrying a CDR of the invention will generally be of an antibody heavy or light chain sequence or substantial portion thereof in which the CDR is located at a location corresponding to the CDR of naturally occurring VH and VL antibody variable domains encoded by rearranged immunoglobulin genes. The structures and locations of immunoglobulin variable domains and CDRs may be determined by reference to (Kabat, E. A. et al, Sequences of Proteins of Immunological Interest. 4th Edition. US Department of Health and Human Services. 1987, and updates thereof, now available on the Internet (http://immuno.bme.nwu.edu)).

Various further aspects and embodiments of the present invention will be apparent to those skilled in the art in view of the present disclosure. All documents and database entries mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above and tables described below.

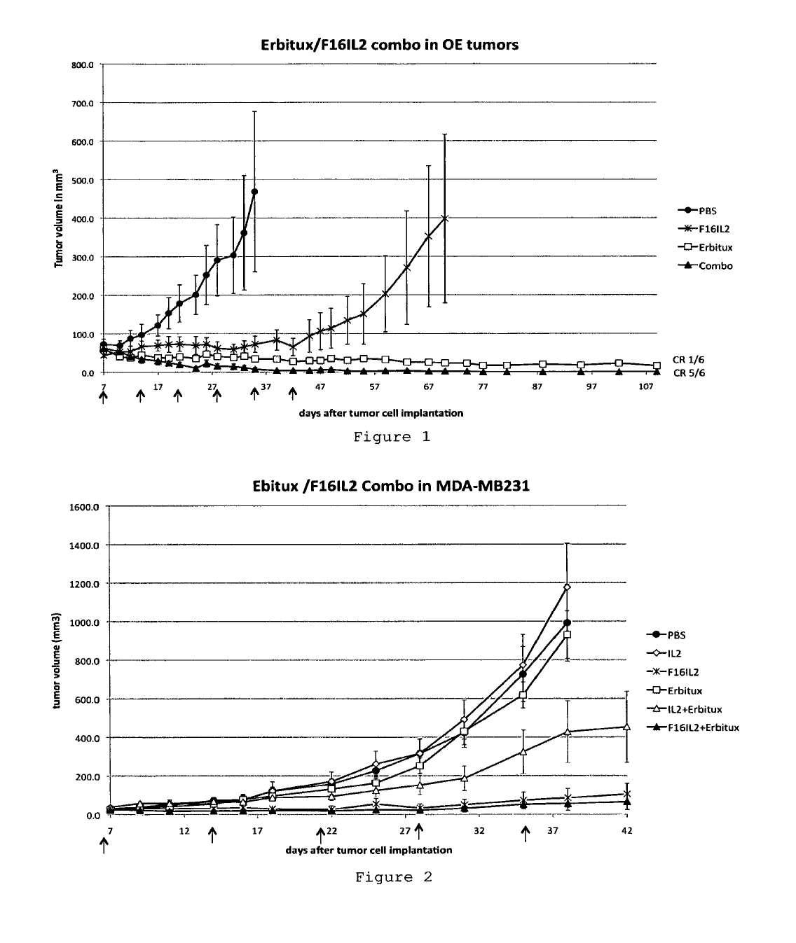

FIG. 1 shows the effect of treatment with F16-IL2 and cetuximab in 10- to 12-week old Balb/c nude female mice injected with 10⁷ HNX-OE human head and neck squamous cell carcinoma (HNSCC) cells.

FIG. 2 shows the effect of treatment with F16-IL2 and cetuximab in 10- to 12-week old Balb/c nude female mice injected with 2×10⁷ MDA-MB-231 human breast cancer cells.

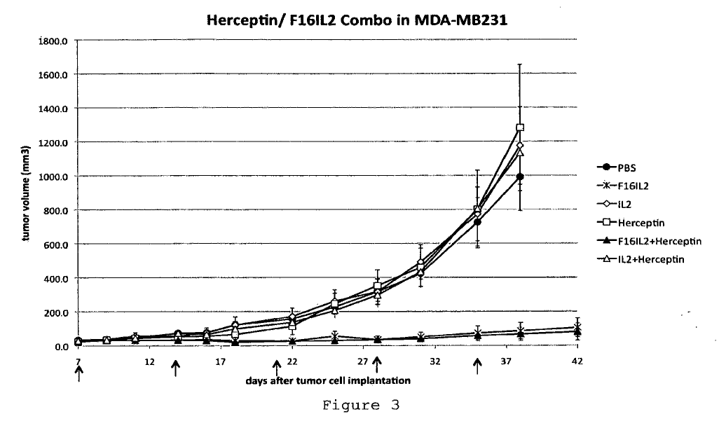

FIG. 3 shows the effect of treatment with F16-IL2 and trastuzumab in 10- to 12-week old Balb/c nude female mice injected with 2×10⁷ MDA-MB-231 human breast cancer cells.

### EXPERIMENTS

1. OE (F16-IL2 in Combination with Erbitux)

Tumor-bearing mice were obtained by injecting 1*10̂7 HNX-OE human HNSCC cells s.c. in 10- to 12-week old Balb/c nude female mice (Charles River Laboratories). Mice were grouped (n=6) 7 days after tumor cell implantation when tumors were clearly palpable and injected i.v. in the lateral tail vein with saline, 20 mg F16-IL2 (corresponding to 6.6 mg IL2), 50 mg/kg cetuximab (Erbitux®) or a combination of both. Injections were given 1× weekly for 6 weeks. Mice were monitored daily and tumor growth was measured three times weekly with a caliper using the following formula: volume=length*width²*0.5. Animals were sacrificed when tumors reached a volume>2000 mm³ or when tumors became necrotic according to Swiss regulations and under a project license granted by the Veterinäramt des Kantons Zürich (169/2008). Tumor sizes are expressed as mean±SE.

2. MDA-MB-231 (F16-IL2 in Combination with Erbitux or Herceptin)

Tumor-bearing mice were obtained by injecting 2*10̂7 MDA-MB-231 human breast cancer cells s.c. in 10- to 12-week old Balb/c nude female mice (Charles River Laboratories). Mice were grouped (n=6) 7 days after tumor cell implantation when tumors were clearly palpable and injected i.v. in the lateral tail vein with saline, 20 mg F16-IL2 (corresponding to 6.6 mg IL2), 6.6 mg recombinant IL2 (Proleukin®), 50 mg/kg cetuximab (Erbitux®), 10 mg/kg trastuzumab (Herceptin®) or the following combinations: F16-IL2 & cetuximab, F16-IL2 & trastuzumab, IL2 & cetuximab, IL2 & trastuzumab. Injections were given 1× weekly for 5 weeks. Mice were monitored daily and tumor growth was measured three times weekly with a caliper using the following formula: volume=length*width²*0.5. Animals were sacrificed when tumors reached a volume>2000 mm³ or when tumors became necrotic according to Swiss regulations and under a project license granted by the Veterinäramt des Kantons Zürich (169/2008). Tumor sizes are expressed as mean±SE.

### Results

1. OE (F16-IL2 in Combination with Erbitux)

At day 81 5 of 6 mice had a complete response in the combination group (FIG. 1; filled triangles) whereas only 1 complete response was seen in the Erbitux group (FIG. 1; empty squares). Students t-test shows that the combination therapy of Erbitux and F16IL2 is significantly better than therapy with Erbitux alone (p=0.0027).

2. MDA-MB-231 (F16-IL2 in Combination with Erbitux or Herceptin)

FIG. 2 shows that treatment of MDA-MB-231 mice with the combination therapy of Erbitux and F16IL2 (FIG. 2; filled triangles) is significantly better than therapy with either Erbitux alone (FIG. 2; empty squares) or F16IL2 alone (FIG. 2; crosses) or combination therapy with Erbitux and IL2 (FIG. 2; empty triangles).

FIG. 3 shows that treatment of MDA-MB-231 mice with the combination therapy of Herceptin and F16IL2 (FIG. 3; filled triangles) is significantly better than therapy with either Herceptin alone (FIG. 2; empty squares) or F16IL2 alone (FIG. 3; crosses) or combination therapy with Herceptin and IL2 (FIG. 3; empty triangles).

### REFERENCES

1. Cartron, G., et al., Therapeutic activity of humanized anti-CD20 monoclonal antibody and polymorphism in IgG Fc receptor FcgammaRIIIa gene. Blood, 2002. 99(3): p. 754-8.
2. Weng, W. K. and R. Levy, Two immunoglobulin G fragment C receptor polymorphisms independently predict response to rituximab in patients with follicular lymphoma. J Clin Oncol, 2003. 21(21): p. 3940-7.
3. Adams, G. P. and L. M. Weiner, Monoclonal antibody therapy of cancer. Nat Biotechnol, 2005. 23(9): p. 1147-57.
4. Umana, P., et al., Engineered glycoforms of an antineuroblastoma IgG1 with optimized antibody-dependent cellular cytotoxic activity. Nat Biotechnol, 1999. 17(2): p. 176-80.
5. Li, H., et al., Optimization of humanized IgGs in glycoengineered Pichia pastoris. Nat Biotechnol, 2006. 24(2): p. 210-5.
6. Zalevsky, J., et al., The impact of Fc engineering on an anti-CD19 antibody: increased Fcgamma receptor affinity enhances B-cell clearing in nonhuman primates. Blood, 2009. 113(16): p. 3735-43.
7. Nimmerjahn, F. and J. V. Ravetch, Antibodies, Fc receptors and cancer. Curr Opin Immunol, 2007. 19(2): p. 239-45.
8. Eisenbeis, C. F., et al., Combination immunotherapy of B-cell non-Hodgkin's lymphoma with rituximab and interleukin-2: a preclinical and phase I study. Clin Cancer Res, 2004. 10(18 Pt 1): p. 6101-10.
9. Gluck, W. L., et al., Phase I studies of interleukin (IL)-2 and rituximab in B-cell non-hodgkin's lymphoma: IL-2 mediated natural killer cell expansion correlations with clinical response. Clin Cancer Res, 2004. 10(7): p. 2253-64.
10. Fleming, G. F., et al., A phase I trial of escalating doses of trastuzumab combined with daily subcutaneous interleukin 2: report of cancer and leukemia group B 9661. Clin Cancer Res, 2002. 8(12): p. 3718-27.
11. Repka, T., et al., Trastuzumab and interleukin-2 in HER2-positive metastatic breast cancer: a pilot study. Clin Cancer Res, 2003. 9(7): p. 2440-6.
12. Hara, M., et al., Interleukin-2 potentiation of cetuximab antitumor activity for epidermal growth factor receptor-overexpressing gastric cancer xenografts through antibody-dependent cellular cytotoxicity. Cancer Sci, 2008. 99(7): p. 1471-8.
13. Schliemann, C., et al., Complete eradication of human B-cell lymphoma xenografts using rituximab in combination with the immunocytokine L19-IL2. Blood, 2009. 113(10): p. 2275-83.
14. Carter, P. J., Potent antibody therapeutics by design. Nat Rev Immunol, 2006. 6(5): p. 343-57.
15. Schrama, D., R. A. Reisfeld, and J. C. Becker, Antibody targeted drugs as cancer therapeutics. Nat Rev Drug Discov, 2006. 5(2): p. 147-59.
16. Neri, D. and R. Bicknell, Tumour vascular targeting. Nat Rev Cancer, 2005. 5(6): p. 436-46.
17. Kaspar, M., L. Zardi, and D. Neri, Fibronectin as target for tumor therapy. Int J Cancer, 2006. 118(6): p. 1331-9.
18. Schliemann, C. and D. Neri, Antibody-based targeting of the tumor vasculature. Biochim Biophys Acta, 2007. 1776(2): p. 175-92.
19. Sauer, S., et al., Expression of the oncofetal ED-B-containing fibronectin isoform in hematologic tumors enables ED-B-targeted 1311-L19SIP radioimmunotherapy in Hodgkin lymphoma patients. Blood, 2009. 113(10): p. 2265-74.
20. Santimaria, M., et al., Immunoscintigraphic detection of the ED-B domain of fibronectin, a marker of angiogenesis, in patients with cancer. Clin Cancer Res, 2003. 9(2): p. 571-9.
21. Rybak, J. N., et al., Ligand-Based Vascular Targeting of Disease. ChemMedChem, 2007. 2(1): p. 22-40.
22. Villa, A., et al., A high-affinity human monoclonal antibody specific to the alternatively spliced EDA domain of fibronectin efficiently targets tumor neo-vasculature in vivo. Int J Cancer, 2008. 122(11): p. 2405-13.
23. Rybak, J. N., et al., The extra-domain A of fibronectin is a vascular marker of solid tumors and metastases. Cancer Res, 2007. 67(22): p. 10948-57.
24. Brack, S. S., et al. Tumor-targeting properties of novel antibodies specific to the large isoform of tenascin-C. Clin Cancer Res, 2006. 12(10): p. 3200-8.
25. Marlind, J., et al., Antibody-mediated delivery of interleukin-2 to the stroma of breast cancer strongly enhances the potency of chemotherapy. Clin Cancer Res, 2008. 14(20): p. 6515-24.
26. Roda, J. M., et al., The activation of natural killer cell effector functions by cetuximab-coated, epidermal growth factor receptor positive tumor cells is enhanced by cytokines. Clin Cancer Res, 2007. 13(21): p. 6419-28.
27. Baselga J and Swain S Novel anticancer targets: revisiting ERBB2 and discovering ERBB3. Nat. Rev. Cancer 2009 Jun. 18 Epub

Sequences
SEQ ID NO: 1.
4A1-F16 VH domain nucleotide sequence
GAG GTG CAG CTG TTG GAG TCT GGG GGA GGC TTG GTA CAG CCT GGG GGG TCC
CTG AGA CTC TCC TGT GCA GCC TCT GGA TTC ACC TTT AGC CGG TAT GGT GCG
AGC TGG GTC CGC CAG GCT CCA GGG AAG GGG CTG GAG TGG GTC TCA GCT ATT
AGT GGT AGT GGT GGT AGC ACA TAC TAC GCA GAC TCC GTG AAG GGC CGG TTC
ACC ATC TCC AGA GAC AAT TCC AAG AAC ACG CTG TAT CTG CAA ATG AAC AGC
CTG AGA GCC GAG GAC ACG GCC GTA TAT TAC TGT GCG AAA GCG CAT AAT GCT
TTT GAC TAC TGG GGC CAG GGA ACC CTG GTC ACC GTG TCG AGA
SEQ ID NO: 2
4A1-F16 VH domain amino acid sequence
EVQLLESGGG LVQPGGSLRL SCAASGFTFS RYGMSWVRQA PGKGLEWVSA ISGSGGSTYY
ADSVKGRFTI SRDNSKNTLY LQMNSLRAED TAVYYCAKAH NAFDYWGQGT LVTVSR
SEQ ID NO: 3
4A1-F16 VL domain nucleotide sequence
TCG TCT GAG CTG ACT CAG GAC CCT GCT GTG TCT GTG GCC TTG GGA CAG ACA
GTC AGG ATC ACA TGC CAA GGA GAC AGC CTC AGA AGC TAT TAT GCA AGC TGG
TAC CAG CAG AAG CCA GGA CAG GCC CCT GTA CTT GTC ATC TAT GGT AAA AAC
AAC CGG CCC TCA GGG ATC CCA GAC CGA TTC TCT GGC TCC AGC TCA GGA AAC
ACA GCT TCC TTG ACC ATC ACT GGG GCT CAG GCG GAA GAT GAG GCT GAC TAT
TAC TGT AAC TCC TCT GTT TAT ACT ATG CCG CCC GTG GTA TTC GGC GGA GGG
ACC AAG CTG ACC GTC CTA GGC
SEQ ID NO: 4
4A1-F16 VL domain amino acid sequence
SSELTQDPAV SVALGQTVRI TCQGDSLRSY YASWYQQKPG QAPVLVIYGK NNRPSGIPDR
FSGSSSGNTA SLTITGAQAE DEADYYCNSS VYTMPPVVFG GGTKLTVL
SEQ ID NO: 5
4A1-F16 VH CDR1 amino acid sequence
RYGMS
SEQ ID NO: 6
4A1-F16 VH CDR2 amino acid sequence
AISGSGGSTYYADSVKG
SEQ ID NO: 7
4A1-F16 VH CDR3 amino acid sequence
AHNAFDY
SEQ ID NO: 8
4A1-F16 VL CDR1 amino acid sequence
QGDSLRSYYAS
SEQ ID NO: 9
4A1-F16 VL CDR2 amino acid sequence
GKNNRPS
SEQ ID NO: 10
4A1-F16 VL CDR3 amino acid sequence
NSSVYTMPPVV
SEQ ID NO: 11
hIL2 precursor sequence (mature hIL2: residues 23-153)
MYRMQLLSCI ALSLALVTNS APTSSSTKKT QLQLEHLLLD LQMILNGINN YKNPKLTRML
TFKFYMPKKA TELKHLQCLE EELKPLEEVL NLAQSKNFHL RPRDLISNIN VIVLELKGSE
TTFMCEYADE TATIVEFLNR WITFCQSIIS TLT
SEQ ID NO: 12
Peptide linker amino acid sequence
GGGGSGGGGSGGGG
SEQ ID NO: 13
Peptide linker amino acid sequence
GGGGSGGGGSGGGGS
SEQ ID NO: 14
Peptide linker amino acid sequence
SSSSGSSSSGSSSSG
SEQ ID NO: 15
Peptide linker amino acid sequence
GSGSAGSGSAGSGSA
SEQ ID NO: 16
Peptide linker amino acid sequence
GGSGGGGSGGGGSGG
SEQ ID NO: 17
Peptide linker amino acid sequence
GGGSGGGSGG

1. A method of treating cancer comprising:
administering an anti-ErbB antibody and an antibody-interleukin 2 (IL2) conjugate to an individual in need thereof,
wherein the antibody-IL2 conjugate comprises IL2 conjugated to an antibody which specifically binds to tenascin-C.

2. A method according to claim 1 wherein the antibody in the said conjugate specifically binds to the tenascin-C large isoform.

3. A method according to claim 2 wherein the antibody in said conjugate specifically binds to the A1 domain of tenascin-C large isoform.

4. A method according to claim 3 wherein the antibody in said conjugate competes for binding to tenascin-C large isoform with an antibody comprising the 4A1-F16 VH domain of SEQ ID NO: 2 and the 4A1-F16 VL domain of SEQ ID NO: 4.

5. A method according to claim 4 wherein the antibody in said conjugate comprises an antibody antigen binding site comprising a VH domain and a VL domain,
the VH domain comprising a VH CDR1 of SEQ ID NO: 5, a VH CDR2 of SEQ ID NO: 6 and a VH CDR3 of SEQ ID NO: 7; and
the VL domain comprising a VL CDR1 of SEQ ID NO: 8, a VL CDR2 of SEQ ID NO: 9 and a VL CDR3 of SEQ ID NO: 10.

6. A method according to claim 5 wherein the antibody comprises an antibody antigen binding site comprising the 4A1-F16 VH domain of SEQ ID NO: 2 and the 4A1-F16 VL domain of SEQ ID NO: 4.

7. A method according to claim 4 wherein the anti-ErbB antibody is an anti-EGFR antibody.

8. A method according to claim 7 wherein the anti-EGFR antibody is cetuximab.

9. A method according to claim 8 wherein the cancer is colorectal cancer, head and neck cancer or non-small cell lung cancer.

10. A method according to claim 4 wherein the anti-ErbB antibody is an anti-HER2 antibody.

11. A method according to claim 10 wherein the anti-HER2 antibody is trastuzumab.

12. A method according to claim 10 wherein the cancer is HER2 over expressing breast cancer.

13-29. (canceled)

30. A kit for use in the treatment of cancer comprising a combination of anti-ErbB antibody and an antibody-IL2 conjugate, said conjugate comprising interleukin 2 (IL2) conjugated to an antibody which specifically binds to tenascin-C.

31. The kit as claimed in claim 30, wherein the antibody in said conjugate competes for binding to tenascin-C large isoform with an antibody comprising the 4A1-F16 VH domain of SEQ ID NO: 2 and the 4A1-F16 VL domain of SEQ ID NO: 4 and said anti-ErbB antibody is cetuximab or trastuzumab.

32. The kit as claimed in claim 30, wherein the antibody in said conjugate comprises an antibody antigen binding site comprising a VH domain and a VL domain,
the VH domain comprising a VH CDR1 of SEQ ID NO: 5, a VH CDR2 of SEQ ID NO: 6 and a VH CDR3 of SEQ ID NO: 7; and
the VL domain comprising a VL CDR1 of SEQ ID NO: 8, a VL CDR2 of SEQ ID NO: 9 and a VL CDR3 of SEQ ID NO: 10 and said anti-ErbB antibody is cetuximab or trastuzumab.

33. The kit as claimed in claim 30, wherein the antibody in said conjugate comprises an antibody antigen binding site comprising the 4A1-F16 VH domain of SEQ ID NO: 2 and the 4A1-F16 VL domain of SEQ ID NO: 4 and said anti-ErbB antibody is cetuximab or trastuzumab.

34. A method according to claim 5, wherein the anti-ErbB antibody is cetuximab or trastuzumab and the cancer is selected from the group consisting of colorectal cancer, head and neck cancer or non-small cell lung cancer.

35. A method according to claim 6, wherein the anti-ErbB antibody is cetuximab or trastuzumab and the cancer is selected from the group consisting of colorectal cancer, head and neck cancer or non-small cell lung cancer.


SEQUENCE LISTING (21 sequences)

SEQ ID NO: 1 - DNA, 348 bp, Artificial sequence
  Synthetic sequence 4A1-F16 VH domain nucleotide sequence
gaggtgcagctgttggagtctgggggaggcttggtacagcctggggggtccctgagactctcctgtgcagcctctggattcacctttagccggtatggtgcgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagcgcataatgcttttgactactggggccagggaaccctggtcaccgtgtcgaga

SEQ ID NO: 2 - protein, 116 aa, Artificial sequence
  Synthetic sequence 4A1-F16 VH domain amino acid sequence
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSR

SEQ ID NO: 3 - DNA, 327 bp, Artificial sequence
  Synthetic sequence 4A1-F16 VL domain nucleotide sequence
tcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcctctgtttatactatgccgcccgtggtattcggcggagggaccaagctgaccgtcctaggc

SEQ ID NO: 4 - protein, 108 aa, Artificial sequence
  Synthetic sequence 4A1-F16 VL domain amino acid sequence
SSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVL

SEQ ID NO: 5 - protein, 5 aa, Artificial sequence
  Synthetic sequence 4A1-F16 VH CDR1 amino acid sequence
RYGMS

SEQ ID NO: 6 - protein, 17 aa, Artificial sequence
  Synthetic sequence 4A1-F16 VH CDR2 amino acid sequence
AISGSGGSTYYADSVKG

SEQ ID NO: 7 - protein, 7 aa, Artificial sequence
  Synthetic sequence 4A1-F16 VH CDR3 amino acid sequence
AHNAFDY

SEQ ID NO: 8 - protein, 11 aa, Artificial sequence
  Synthetic sequence 4A1-F16 VL CDR1 amino acid sequence
QGDSLRSYYAS

SEQ ID NO: 9 - protein, 7 aa, Artificial sequence
  Synthetic sequence 4A1-F16 VL CDR2 amino acid sequence
GKNNRPS

SEQ ID NO: 10 - protein, 11 aa, Artificial sequence
  Synthetic sequence 4A1-F16 VL CDR3 amino acid sequence
NSSVYTMPPVV

SEQ ID NO: 11 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 14 aa, Artificial sequence
  Synthetic sequence Peptide linker amino acid sequence
GGGGSGGGGSGGGG

SEQ ID NO: 13 - protein, 15 aa, Artificial sequence
  Synthetic sequence Peptide linker amino acid sequence
GGGGSGGGGSGGGGS

SEQ ID NO: 14 - protein, 15 aa, Artificial sequence
  Synthetic sequence Peptide linker amino acid sequence
SSSSGSSSSGSSSSG

SEQ ID NO: 15 - protein, 15 aa, Artificial sequence
  Synthetic sequence Peptide linker amino acid sequence
GSGSAGSGSAGSGSA

SEQ ID NO: 16 - protein, 15 aa, Artificial sequence
  Synthetic sequence Peptide linker amino acid sequence
GGSGGGGSGGGGSGG

SEQ ID NO: 17 - protein, 10 aa, Artificial sequence
  Synthetic sequence Peptide linker amino acid sequence
GGGSGGGSGG

SEQ ID NO: 18 - protein, 5 aa, Artificial sequence
  Synthetic sequence Linker motif
GGGGS

SEQ ID NO: 19 - protein, 5 aa, Artificial sequence
  Synthetic sequence Linker motif
SSSSG

SEQ ID NO: 20 - protein, 5 aa, Artificial sequence
  Synthetic sequence Linker motif
GSGSA

SEQ ID NO: 21 - protein, 5 aa, Artificial sequence
  Synthetic sequence Linker motif
GGSGG